SEQUENCE LISTING (145 sequences)

SEQ ID NO: 1 - protein, 12 aa, Unknown
  source: /note=“Description of Unknown Helical peptide domain sequence”
KRIVQRIKDFLR

SEQ ID NO: 2 - protein, 12 aa, Unknown
  source: /note=“Description of Unknown Helical peptide domain sequence”
RLFDKIRQVIRK

SEQ ID NO: 3 - protein, 12 aa, Unknown
  source: /note=“Description of Unknown Helical peptide domain sequence”
KLIKLIKKILKK

SEQ ID NO: 4 - protein, 12 aa, Unknown
  source: /note=“Description of Unknown Helical peptide domain sequence”
KKLIKKILKILK

SEQ ID NO: 5 - protein, 12 aa, Unknown
  source: /note=“Description of Unknown Helical peptide domain sequence”
KEIVRRIEKFLR

SEQ ID NO: 6 - protein, 12 aa, Unknown
  source: /note=“Description of Unknown Helical peptide domain sequence”
RLFKEIRRVIEK

SEQ ID NO: 7 - protein, 12 aa, Unknown
  source: /note=“Description of Unknown Helical peptide domain sequence”
KRIVERIEKFLR

SEQ ID NO: 8 - protein, 12 aa, Unknown
  source: /note=“Description of Unknown Helical peptide domain sequence”
RLFKEIREVIRK

SEQ ID NO: 9 - protein, 12 aa, Unknown
  source: /note=“Description of Unknown Helical peptide domain sequence”
KRLVQRLKDFLR

SEQ ID NO: 10 - protein, 12 aa, Unknown
  source: /note=“Description of Unknown Helical peptide domain sequence”
RLFDKLRQVLRK

SEQ ID NO: 11 - protein, 12 aa, Unknown
  source: /note=“Description of Unknown Helical peptide domain sequence”
KRLIQRKRLIQR

SEQ ID NO: 12 - protein, 12 aa, Unknown
  source: /note=“Description of Unknown Helical peptide domain sequence”
RQILRKRQILRK

SEQ ID NO: 13 - protein, 11 aa, Unknown
  source: /note=“Description of Unknown Helical peptide domain sequence”
LIKLIKKILKK

SEQ ID NO: 14 - protein, 11 aa, Unknown
  source: /note=“Description of Unknown Helical peptide domain sequence”
KKLIKKILKIL

SEQ ID NO: 15 - protein, 11 aa, Unknown
  source: /note=“Description of Unknown Helical peptide domain sequence”
KKLAKEILKAL

SEQ ID NO: 16 - protein, 11 aa, Unknown
  source: /note=“Description of Unknown Helical peptide domain sequence”
LAKLIEKALKK

SEQ ID NO: 17 - protein, 11 aa, Unknown
  source: /note=“Description of Unknown Helical peptide domain sequence”
RRLIRRILRIL

SEQ ID NO: 18 - protein, 11 aa, Unknown
  source: /note=“Description of Unknown Helical peptide domain sequence”
LIRLIRRILRR

SEQ ID NO: 19 - protein, 11 aa, Unknown
  source: /note=“Description of Unknown Helical peptide domain sequence”
LIRLLRRILRR

SEQ ID NO: 20 - protein, 11 aa, Unknown
  source: /note=“Description of Unknown Helical peptide domain sequence”
RRLIRRLLRIL

SEQ ID NO: 21 - protein, 12 aa, Unknown
  source: /note=“Description of Unknown Helical peptide domain sequence”
LLIKLIKKILKK

SEQ ID NO: 22 - protein, 12 aa, Unknown
  source: /note=“Description of Unknown Helical peptide domain sequence”
KKLIKKILKILL

SEQ ID NO: 23 - protein, 13 aa, Unknown
  source: /note=“Description of Unknown Helical peptide domain sequence”
HPLIKLIKKILKK

SEQ ID NO: 24 - protein, 13 aa, Unknown
  source: /note=“Description of Unknown Helical peptide domain sequence”
KKLIKKILKILPH

SEQ ID NO: 25 - protein, 13 aa, Unknown
  source: /note=“Description of Unknown Helical peptide domain sequence”
GRLIKLIKKILKK

SEQ ID NO: 26 - protein, 13 aa, Unknown
  source: /note=“Description of Unknown Helical peptide domain sequence”
KKLIKKILKILRG

SEQ ID NO: 27 - protein, 12 aa, Unknown
  source: /note=“Description of Unknown Helical peptide domain sequence”
KLIRLIREILRR

SEQ ID NO: 28 - protein, 12 aa, Unknown
  source: /note=“Description of Unknown Helical peptide domain sequence”
RRLIERILRILK

SEQ ID NO: 29 - protein, 12 aa, Unknown
  source: /note=“Description of Unknown Helical peptide domain sequence”
KEIVRRIKEFLR

SEQ ID NO: 30 - protein, 12 aa, Unknown
  source: /note=“Description of Unknown Helical peptide domain sequence”
RLFEKIRRVIEK

SEQ ID NO: 31 - protein, 11 aa, Unknown
  source: /note=“Description of Unknown Helical peptide domain sequence”
LIKLCKKILKK

SEQ ID NO: 32 - protein, 11 aa, Unknown
  source: /note=“Description of Unknown Helical peptide domain sequence”
KKLIKKCLKIL

SEQ ID NO: 33 - protein, 16 aa, Unknown
  source: /note=“Description of Unknown Helical peptide domain sequence”
KVLSRVHAALKSIFDL

SEQ ID NO: 34 - protein, 16 aa, Unknown
  source: /note=“Description of Unknown Helical peptide domain sequence”
LDFISKLAAHVRSLVK

SEQ ID NO: 35 - protein, 3 aa, Artificial Sequence
  source: /note=“Description of Artificial Sequence Synthetic peptide”
AAA

SEQ ID NO: 36 - protein, 10 aa, Artificial Sequence
  source: /note=“Description of Artificial Sequence Synthetic peptide”
GGGSSGGGSG

SEQ ID NO: 37 - protein, 4 aa, Artificial Sequence
  source: /note=“Description of Artificial Sequence Synthetic peptide”
GPGR

SEQ ID NO: 38 - protein, 8 aa, Artificial Sequence
  source: /note=“Description of Artificial Sequence Synthetic peptide”
RDTPVVKS

SEQ ID NO: 39 - protein, 27 aa, Artificial Sequence
  source: /note=“Description of Artificial Sequence Synthetic peptide”
AKDGIPAPTNYHKKHRAPVSCTGPAKM

SEQ ID NO: 40 - protein, 7 aa, Artificial Sequence
  source: /note=“Description of Artificial Sequence Synthetic peptide”
GSTAPPA

SEQ ID NO: 41 - protein, 25 aa, Artificial Sequence
  source: /note=“Description of Artificial Sequence Synthetic peptide”
RANATTLPKYYQNSRHPVSCTDPSK

SEQ ID NO: 42 - protein, 2 aa, Artificial Sequence
  source: /note=“Description of Artificial Sequence Synthetic peptide”
RW

SEQ ID NO: 43 - protein, 3 aa, Artificial Sequence
  source: /note=“Description of Artificial Sequence Synthetic peptide”
SRD

SEQ ID NO: 44 - protein, 7 aa, Artificial Sequence
  source: /note=“Description of Artificial Sequence Synthetic peptide”
GSTAPPA

SEQ ID NO: 45 - protein, 14 aa, Artificial Sequence
  source: /note=“Description of Artificial Sequence Synthetic peptide”
GSTAPPAGSTAPPA

SEQ ID NO: 46 - protein, 15 aa, Artificial Sequence
  source: /note=“Description of Artificial Sequence Synthetic peptide”
QASHTCVCEFNCAPL

SEQ ID NO: 47 - protein, 27 aa, Artificial Sequence
  source: /note=“Description of Artificial Sequence Synthetic peptide”
ARKKASIPNYYNSNLQPPVFCSDQSKM

SEQ ID NO: 48 - protein, 14 aa, Artificial Sequence
  source: /note=“Description of Artificial Sequence Synthetic peptide”
YEQGAGRGSTAPPA

SEQ ID NO: 49 - protein, 4 aa, Artificial Sequence
  source: /note=“Description of Artificial Sequence Synthetic peptide”
GSTA

SEQ ID NO: 50 - protein, 11 aa, Artificial Sequence
  source: /note=“Description of Artificial Sequence Synthetic peptide”
GGGSGGGTDGR

SEQ ID NO: 51 - protein, 28 aa, Artificial Sequence
  source: /note=“Description of Artificial Sequence Synthetic peptide”
KRIVQRIKDFLRGPGRKRIVQRIKDFLR

SEQ ID NO: 52 - protein, 28 aa, Artificial Sequence
  source: /note=“Description of Artificial Sequence Synthetic peptide”
KRIVQRIKDFLRGPGRRLFDKIRQVIRK

SEQ ID NO: 53 - protein, 28 aa, Artificial Sequence
  source: /note=“Description of Artificial Sequence Synthetic peptide”
RLFDKIRQVIRKGPGRKRIVQRIKDFLR

SEQ ID NO: 54 - protein, 28 aa, Artificial Sequence
  source: /note=“Description of Artificial Sequence Synthetic peptide”
RLFDKIRQVIRKGPGRRLFDKIRQVIRK

SEQ ID NO: 55 - protein, 28 aa, Artificial Sequence
  source: /note=“Description of Artificial Sequence Synthetic peptide”
KLIKLIKKILKKGPGRKLIKLIKKILKK

SEQ ID NO: 56 - protein, 28 aa, Artificial Sequence
  source: /note=“Description of Artificial Sequence Synthetic peptide”
KLIKLIKKILKKGPGRKKLIKKILKILK

SEQ ID NO: 57 - protein, 28 aa, Artificial Sequence
  source: /note=“Description of Artificial Sequence Synthetic peptide”
KKLIKKILKILKGPGRKLIKLIKKILKK

SEQ ID NO: 58 - protein, 28 aa, Artificial Sequence
  source: /note=“Description of Artificial Sequence Synthetic peptide”
KKLIKKILKILKGPGRKKLIKKILKILK

SEQ ID NO: 59 - protein, 28 aa, Artificial Sequence
  source: /note=“Description of Artificial Sequence Synthetic peptide”
KEIVRRIEKFLRGPGRKRIVERIEKFLR

SEQ ID NO: 60 - protein, 28 aa, Artificial Sequence
  source: /note=“Description of Artificial Sequence Synthetic peptide”
KEIVRRIEKFLRGPGRKEIVRRIEKFLR

SEQ ID NO: 61 - protein, 28 aa, Artificial Sequence
  source: /note=“Description of Artificial Sequence Synthetic peptide”
KEIVRRIEKFLRGPGRRLFKEIRRVIEK

SEQ ID NO: 62 - protein, 28 aa, Artificial Sequence
  source: /note=“Description of Artificial Sequence Synthetic peptide”
RLFKEIRRVIEKGPGRKRIVERIEKFLR

SEQ ID NO: 63 - protein, 28 aa, Artificial Sequence
  source: /note=“Description of Artificial Sequence Synthetic peptide”
RLFKEIRRVIEKGPGRRLFKEIRRVIEK

SEQ ID NO: 64 - protein, 28 aa, Artificial Sequence
  source: /note=“Description of Artificial Sequence Synthetic peptide”
KRIVERIEKFLRGPGRKEIVRRIEKFLR

SEQ ID NO: 65 - protein, 28 aa, Artificial Sequence
  source: /note=“Description of Artificial Sequence Synthetic peptide”
KRIVERIEKFLRGPGRKRIVERIEKFLR

SEQ ID NO: 66 - protein, 28 aa, Artificial Sequence
  source: /note=“Description of Artificial Sequence Synthetic peptide”
KRIVERIEKFLRGPGRRLFKEIREVIRK

SEQ ID NO: 67 - protein, 28 aa, Artificial Sequence
  source: /note=“Description of Artificial Sequence Synthetic peptide”
RLFKEIREVIRKGPGRKEIVRRIEKFLR

SEQ ID NO: 68 - protein, 28 aa, Artificial Sequence
  source: /note=“Description of Artificial Sequence Synthetic peptide”
RLFKEIREVIRKGPGRRLFKEIREVIRK

SEQ ID NO: 69 - protein, 28 aa, Artificial Sequence
  source: /note=“Description of Artificial Sequence Synthetic peptide”
KRLVQRLKDFLRGPGRKRLVQRLKDFLR

SEQ ID NO: 70 - protein, 28 aa, Artificial Sequence
  source: /note=“Description of Artificial Sequence Synthetic peptide”
KRLVQRLKDFLRGPGRRLFDKLRQVLRK

SEQ ID NO: 71 - protein, 28 aa, Artificial Sequence
  source: /note=“Description of Artificial Sequence Synthetic peptide”
RLFDKLRQVLRKGPGRKRLVQRLKDFLR

SEQ ID NO: 72 - protein, 28 aa, Artificial Sequence
  source: /note=“Description of Artificial Sequence Synthetic peptide”
RLFDKLRQVLRKGPGRRLFDKLRQVLRK

SEQ ID NO: 73 - protein, 28 aa, Artificial Sequence
  source: /note=“Description of Artificial Sequence Synthetic peptide”
KRLIQRKRLIQRGPGRKRLIQRKRLIQR

SEQ ID NO: 74 - protein, 28 aa, Artificial Sequence
  source: /note=“Description of Artificial Sequence Synthetic peptide”
KRLIQRKRLIQRGPGRRQILRKRQILRK

SEQ ID NO: 75 - protein, 28 aa, Artificial Sequence
  source: /note=“Description of Artificial Sequence Synthetic peptide”
RQILRKRQILRKGPGRKRLIQRKRLIQR

SEQ ID NO: 76 - protein, 28 aa, Artificial Sequence
  source: /note=“Description of Artificial Sequence Synthetic peptide”
RQILRKRQILRKGPGRRQILRKRQILRK

SEQ ID NO: 77 - protein, 26 aa, Artificial Sequence
  source: /note=“Description of Artificial Sequence Synthetic peptide”
LIKLIKKILKKGPGRLIKLIKKILKK

SEQ ID NO: 78 - protein, 26 aa, Artificial Sequence
  source: /note=“Description of Artificial Sequence Synthetic peptide”
LIKLIKKILKKGPGRKKLIKKILKIL

SEQ ID NO: 79 - protein, 26 aa, Artificial Sequence
  source: /note=“Description of Artificial Sequence Synthetic peptide”
KKLIKKILKILGPGRLIKLIKKILKK

SEQ ID NO: 80 - protein, 26 aa, Artificial Sequence
  source: /note=“Description of Artificial Sequence Synthetic peptide”
KKLIKKILKILGPGRKKLIKKILKIL

SEQ ID NO: 81 - protein, 26 aa, Artificial Sequence
  source: /note=“Description of Artificial Sequence Synthetic peptide”
KKLAKEILKALGPGRKKLAKEILKAL

SEQ ID NO: 82 - protein, 26 aa, Artificial Sequence
  source: /note=“Description of Artificial Sequence Synthetic peptide”
KKLAKEILKALGPGRLAKLIEKALKK

SEQ ID NO: 83 - protein, 26 aa, Artificial Sequence
  source: /note=“Description of Artificial Sequence Synthetic peptide”
LAKLIEKALKKGPGRKKLAKEILKAL

SEQ ID NO: 84 - protein, 26 aa, Artificial Sequence
  source: /note=“Description of Artificial Sequence Synthetic peptide”
LAKLIEKALKKGPGRLAKLIEKALKK

SEQ ID NO: 85 - protein, 26 aa, Artificial Sequence
  source: /note=“Description of Artificial Sequence Synthetic peptide”
RRLIRRILRILGPGRRRLIRRILRIL

SEQ ID NO: 86 - protein, 26 aa, Artificial Sequence
  source: /note=“Description of Artificial Sequence Synthetic peptide”
RRLIRRILRILGPGRLIRLIRRILRR

SEQ ID NO: 87 - protein, 26 aa, Artificial Sequence
  source: /note=“Description of Artificial Sequence Synthetic peptide”
LIRLIRRILRRGPGRRRLIRRILRIL

SEQ ID NO: 88 - protein, 26 aa, Artificial Sequence
  source: /note=“Description of Artificial Sequence Synthetic peptide”
LIRLIRRILRRGPGRLIRLIRRILRR

SEQ ID NO: 89 - protein, 26 aa, Artificial Sequence
  source: /note=“Description of Artificial Sequence Synthetic peptide”
LIRLLRRILRRGPGRLIRLLRRILRR

SEQ ID NO: 90 - protein, 26 aa, Artificial Sequence
  source: /note=“Description of Artificial Sequence Synthetic peptide”
LIRLLRRILRRGPGRRRLIRRLLRIL

SEQ ID NO: 91 - protein, 26 aa, Artificial Sequence
  source: /note=“Description of Artificial Sequence Synthetic peptide”
RRLIRRLLRILGPGRLIRLLRRILRR

SEQ ID NO: 92 - protein, 26 aa, Artificial Sequence
  source: /note=“Description of Artificial Sequence Synthetic peptide”
RRLIRRLLRILGPGRRRLIRRLLRIL

SEQ ID NO: 93 - protein, 28 aa, Artificial Sequence
  source: /note=“Description of Artificial Sequence Synthetic peptide”
LLIKLIKKILKKGPGRLLIKLIKKILKK

SEQ ID NO: 94 - protein, 28 aa, Artificial Sequence
  source: /note=“Description of Artificial Sequence Synthetic peptide”
LLIKLIKKILKKGPGRKKLIKKILKILL

SEQ ID NO: 95 - protein, 28 aa, Artificial Sequence
  source: /note=“Description of Artificial Sequence Synthetic peptide”
KKLIKKILKILLGPGRLLIKLIKKILKK

SEQ ID NO: 96 - protein, 28 aa, Artificial Sequence
  source: /note=“Description of Artificial Sequence Synthetic peptide”
KKLIKKILKILLGPGRKKLIKKILKILL

SEQ ID NO: 97 - protein, 30 aa, Artificial Sequence
  source: /note=“Description of Artificial Sequence Synthetic polypeptide”
HPLIKLIKKILKKGPGRHPLIKLIKKILKK

SEQ ID NO: 98 - protein, 30 aa, Artificial Sequence
  source: /note=“Description of Artificial Sequence Synthetic polypeptide”
HPLIKLIKKILKKGPGRKKLIKKILKILPH

SEQ ID NO: 99 - protein, 30 aa, Artificial Sequence
  source: /note=“Description of Artificial Sequence Synthetic polypeptide”
KKLIKKILKILPHGPGRHPLIKLIKKILKK

SEQ ID NO: 100 - protein, 30 aa, Artificial Sequence
  source: /note=“Description of Artificial Sequence Synthetic polypeptide”
KKLIKKILKILPHGPGRKKLIKKILKILPH

SEQ ID NO: 101 - protein, 30 aa, Artificial Sequence
  source: /note=“Description of Artificial Sequence Synthetic polypeptide”
GRLIKLIKKILKKGPGRGRLIKLIKKILKK

SEQ ID NO: 102 - protein, 30 aa, Artificial Sequence
  source: /note=“Description of Artificial Sequence Synthetic polypeptide”
GRLIKLIKKILKKGPGRKKLIKKILKILRG

SEQ ID NO: 103 - protein, 30 aa, Artificial Sequence
  source: /note=“Description of Artificial Sequence Synthetic polypeptide”
KKLIKKILKILRGGPGRGRLIKLIKKILKK

SEQ ID NO: 104 - protein, 30 aa, Artificial Sequence
  source: /note=“Description of Artificial Sequence Synthetic polypeptide”
KKLIKKILKILRGGPGRKKLIKKILKILRG

SEQ ID NO: 105 - protein, 28 aa, Artificial Sequence
  source: /note=“Description of Artificial Sequence Synthetic peptide”
KLIRLIREILRRGPGRKLIRLIREILRR

SEQ ID NO: 106 - protein, 28 aa, Artificial Sequence
  source: /note=“Description of Artificial Sequence Synthetic peptide”
KLIRLIREILRRGPGRRRLIERILRILK

SEQ ID NO: 107 - protein, 28 aa, Artificial Sequence
  source: /note=“Description of Artificial Sequence Synthetic peptide”
RRLIERILRILKGPGRKLIRLIREILRR

SEQ ID NO: 108 - protein, 28 aa, Artificial Sequence
  source: /note=“Description of Artificial Sequence Synthetic peptide”
RRLIERILRILKGPGRRRLIERILRILK

SEQ ID NO: 109 - protein, 28 aa, Artificial Sequence
  source: /note=“Description of Artificial Sequence Synthetic peptide”
KEIVRRIKEFLRGPGRKEIVRRIKEFLR

SEQ ID NO: 110 - protein, 28 aa, Artificial Sequence
  source: /note=“Description of Artificial Sequence Synthetic peptide”
KEIVRRIKEFLRGPGRRLFEKIRRVIEK

SEQ ID NO: 111 - protein, 28 aa, Artificial Sequence
  source: /note=“Description of Artificial Sequence Synthetic peptide”
RLFEKIRRVIEKGPGRKEIVRRIKEFLR

SEQ ID NO: 112 - protein, 28 aa, Artificial Sequence
  source: /note=“Description of Artificial Sequence Synthetic peptide”
RLFEKIRRVIEKGPGRRLFEKIRRVIEK

SEQ ID NO: 113 - protein, 26 aa, Artificial Sequence
  source: /note=“Description of Artificial Sequence Synthetic peptide”
LIKLCKKILKKGPGRLIKLCKKILKK

SEQ ID NO: 114 - protein, 26 aa, Artificial Sequence
  source: /note=“Description of Artificial Sequence Synthetic peptide”
LIKLCKKILKKGPGRKKLIKKCLKIL

SEQ ID NO: 115 - protein, 26 aa, Artificial Sequence
  source: /note=“Description of Artificial Sequence Synthetic peptide”
KKLIKKCLKILGPGRLIKLCKKILKK

SEQ ID NO: 116 - protein, 26 aa, Artificial Sequence
  source: /note=“Description of Artificial Sequence Synthetic peptide”
KKLIKKCLKILGPGRKKLIKKCLKIL

SEQ ID NO: 117 - protein, 36 aa, Artificial Sequence
  source: /note=“Description of Artificial Sequence Synthetic polypeptide”
KVLSRVHAALKSIFDLGPGRKVLSRVHAALKSIFDL

SEQ ID NO: 118 - protein, 36 aa, Artificial Sequence
  source: /note=“Description of Artificial Sequence Synthetic polypeptide”
KVLSRVHAALKSIFDLGPGRLDFISKLAAHVRSLVK

SEQ ID NO: 119 - protein, 36 aa, Artificial Sequence
  source: /note=“Description of Artificial Sequence Synthetic polypeptide”
LDFISKLAAHVRSLVKGPGRKVLSRVHAALKSIFDL

SEQ ID NO: 120 - protein, 36 aa, Artificial Sequence
  source: /note=“Description of Artificial Sequence Synthetic polypeptide”
LDFISKLAAHVRSLVKGPGRLDFISKLAAHVRSLVK

SEQ ID NO: 121 - protein, 9 aa, Artificial Sequence
  source: /note=“Description of Artificial Sequence Synthetic peptide”
KRIVQRIKD

SEQ ID NO: 122 - protein, 9 aa, Malus domestica
KKIIQRIKD

SEQ ID NO: 123 - protein, 7 aa, Artificial Sequence
  source: /note=“Description of Artificial Sequence Synthetic peptide”
RIKDFLR

SEQ ID NO: 124 - protein, 7 aa, Malus domestica
RIKDFLR

SEQ ID NO: 125 - protein, 10 aa, Artificial Sequence
  source: /note=“Description of Artificial Sequence Synthetic peptide”
VQRIKDFLRG

SEQ ID NO: 126 - protein, 10 aa, Malus domestica
VSRIQDFLRG

SEQ ID NO: 127 - protein, 8 aa, Artificial Sequence
  source: /note=“Description of Artificial Sequence Synthetic peptide”
IKKILKKG

SEQ ID NO: 128 - protein, 8 aa, Malus domestica
IKKILKEG

SEQ ID NO: 129 - protein, 11 aa, Artificial Sequence
  source: /note=“Description of Artificial Sequence Synthetic peptide”
LIKKILKKGPG

SEQ ID NO: 130 - protein, 11 aa, Malus domestica
LKKKLLKEGEG

SEQ ID NO: 131 - protein, 11 aa, Artificial Sequence
  source: /note=“Description of Artificial Sequence Synthetic peptide”
KRIVERIEKFL

SEQ ID NO: 132 - protein, 11 aa, Malus domestica
KREIERREKFL

SEQ ID NO: 133 - protein, 8 aa, Artificial Sequence
  source: /note=“Description of Artificial Sequence Synthetic peptide”
VERIEKFL

SEQ ID NO: 134 - protein, 8 aa, Malus domestica
VVRIVKFL

SEQ ID NO: 135 - protein, 15 aa, Artificial Sequence
  source: /note=“Description of Artificial Sequence Synthetic peptide”
VLSRVHAALKSIFDL

SEQ ID NO: 136 - protein, 15 aa, Malus domestica
VLSRVHSALNDIADM

SEQ ID NO: 137 - protein, 19 aa, Artificial Sequence
  source: /note=“Description of Artificial Sequence Synthetic peptide”
VLSRVHAALKSIFDLGPGR

SEQ ID NO: 138 - protein, 22 aa, Malus domestica
VLSRVHSALNDIADMLPLSPAR

SEQ ID NO: 139 - protein, 12 aa, Artificial Sequence
  source: /note=“Description of Artificial Sequence Synthetic peptide”
RVHAALKSIFDL

SEQ ID NO: 140 - protein, 12 aa, Malus domestica
RVKAQLKSMFDL

SEQ ID NO: 141 - DNA, 966 bp, Erwinia amylovora
aaactgatttaaccttgccactatccttaacaggaagaacgacgtattcacggcttcgcagatccctttcccccctaatccctccagaatttattaacattttccgtcggattgctcatagcagaattctgacagtgatgattttcgcttacttagccataacgcaacaaatatatgccagcggatatccctaaaaacctcagtgcgattacagaattacagaattacagaattacagaattacagaattacagaattatcaaacaggctgcatttaaatacagttaatcttaacacagaatgggacagaacaagaatagatctcacgaaaggaaagacatccggcttctgaaatacgcctgaaaaaatcaagagatactaatcattaattccggtgaaattatttactgatactgatgacggtatcttgtcatcccgtaatcccgggatgaccgtttaatcactccaggggtaattacggcaacaaaaatgatctcttttctgaaggggaaaggtggggcagggaaaaccacaacattcatcaacatcgctaccagcctcgcacgtaaagggcacaaggttgcgatggtcgatactgatcctcagggcagcatcagtaactggtataacgagtcaaaatgtctattcgaccttgcagagacatcatcggaaaaaagaagtttatagcgtgcgtaaatatctgaaaaatcatgatgatgtggtcactgatggtgccgttgccattacctccgcagccgtcatggtgagcgatctggtatgaattccggtaacaccttcactgctcgactttgctgcatatgcgactatcctggcggttgtggaggcaagaaaaaatttacagcccgtcattgcacgttttgtgatcgcctaaaagtggcctccacgcgcacgctggaggttctgcaagcttcgatcgaggaaacagtgtccaggtttaaaaaacgg

SEQ ID NO: 142 - DNA, 17 bp, Artificial Sequence
  source: /note=“Description of Artificial Sequence Synthetic oligonucleotide”
cactgatggtgccgttg

SEQ ID NO: 143 - DNA, 18 bp, Artificial Sequence
  source: /note=“Description of Artificial Sequence Synthetic oligonucleotide”
cgccaggatagtcgcata

SEQ ID NO: 144 - DNA, 22 bp, Artificial Sequence
  source: /note=“Description of Artificial Sequence Synthetic oligonucleotide”
gcaggagttgttctttctttgg

SEQ ID NO: 145 - DNA, 22 bp, Artificial Sequence
  source: /note=“Description of Artificial Sequence Synthetic oligonucleotide”
gccatcgtttctcattgacttg

(19) United States
(12) Patent Application Publication
(10) Pub. No.: US 2023/0147468 A1
(43) Pub. Date: May 11, 2023

(54) RECOMBINANT PEPTIDE TO TREAT FIRE BLIGHT

(71) Applicant: INNATE IMMUNITY LLC, Santa Fe, MN (US)

(72) Inventor: Goutam GUPTA, Santa Fe, NM (US)

(73) Assignee: INNATE IMMUNITY LLC, Santa Fe, NM (US)

(21) Appl. No.: 17/915,301

(22) PCT Filed: Mar. 30, 2021
(86) PCT No.: PCT/US2021/024812

§ 371 Date: Sep. 28, 2022

Related U.S. Application Data
(60) Provisional application No. 63/003,134, filed on Mar. 31, 2020.

(51) Int. Cl. C07K 14/415; A01N 65/34; A01P 1/00

(52) U.S. Cl. CPC C07K 14/415; A01N 65/34; A01P 1/00

(57) ABSTRACT

The present disclosure relates generally to recombination peptides composed of at least two helical domains connected by a linker/turn. The disclosed peptides may have a variety of beneficial agricultural properties and uses, for example, in the treatment and prevention of fire blight in apple and pear trees or other members of the Rosaceae family.

### CROSS REFERENCE STATEMENT

This application claims priority under 35 U.S.C. § 119(e) to U.S. Provisional Application 63/003,134 filed Mar. 31, 2020, and the entire contents of this provisional application is incorporated herein by reference.

### FIELD OF INVENTION

The present disclosure relates generally to recombination peptides composed of at least two helical domains connected by a linker/turn. The disclosed peptides may have a variety of beneficial agricultural properties and uses, for example, in the treatment and prevention of fire blight in apple and pear.

### BACKGROUND

The following discussion is merely provided to aid the reader in understanding the disclosure and is not admitted to describe or constitute prior art thereto.

Fire blight is a contagious disease that affects apples and pears, among other plants in the Rosaceae family. It is a serious concern to apple and pear producers around the world (including Europe, Australia, New Zealand, and Japan, among others), and particularly in North America. The causal pathogen is Erwinia amylovora, a Gram-negative bacterium in the order Enterobacterales.

Most infected leaves and branch tips wilt rapidly and turn brown or black. The leaves die but generally do not drop off, which gives the infected plant a burnt appearance. Trees will also develop reddish water soaked lesions on the bark. On warm days, these lesions may ooze an orange-brown liquid. Fire blight kills individual blossoms, shoots, limbs, and sometimes, the entire infected tree.

Chemical control of fire blight is not always effective, and while pruning can potentially keep an infected tree alive, it does little to prevent further spread of the disease or treat an infected tree.

Accordingly, there is a need in the art for compounds that can be safely and efficiently treat or prevent fire blight in apple and pear trees. The present disclosure fulfills that need.

### SUMMARY

Described herein are novel recombinant peptides and methods of using the same to treat or prevent the agricultural infection fire blight.

In one aspect, the disclosure provides recombinant peptides comprising at least two helical peptide domains connected by a linker domain, wherein the at least two helical domains were isolated or derived from an apple or pear plant.

In some embodiments, the at least two helical domains are about 10 to about 20 amino acids in length. In some embodiments, the linker domain is about 4 amino acids. In some embodiments, the recombinant peptide comprises 28 amino acids or 36 amino acids.

In some embodiments, each helical peptide domain is independently selected from any one of SEQ ID NOs: 1-34. In some embodiments, the at least two helical peptides comprise the same amino acid sequence. In some embodiments, the at least two helical peptides comprise different amino acid sequences. In some embodiments, the linker domain comprises SEQ ID NO: 37.

In some embodiments, the recombinant peptide comprises any one of SEQ ID NOs: 51-120. In some embodiments, the recombinant peptide comprises an amino acid sequence that comprises at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homology to any one of SEQ ID NOs: 51-120.

In some embodiments, the recombinant peptide comprising an amino acid sequence selected from SEQ ID NO: 51, SEQ ID NO: 55, SEQ ID NO: 59, and SEQ ID NO: 117.

The present disclosure also provides formulations comprising a recombinant peptide according to any one of the foregoing aspects or embodiments and an acceptable carrier or diluent. In some embodiments, the carrier is a solid. In some embodiments, the carrier is a liquid, such as a spray or aerosol.

In another aspect, the present disclosure provides methods of treating or preventing a fire blight infection in a plant from the Rosaceae family comprising, applying to a target area on or adjacent to a plant from the Rosaceae family an effective amount of a recombinant peptide or formulation thereof of any of the foregoing aspects or embodiments.

In some embodiments, the target area comprises a plant, the seed of a plant, or a portion of the plant. In some embodiments, the target area is the soil in which a plant from the Rosaceae family is growing, a field that will be planted, or a structure on which a plant is growing.

In some embodiments, applying comprises spraying the target with the recombinant peptide.

In some embodiments, the plant from the Rosaceae family is an apple plant or a pear plant.

In another aspect, the present disclosure provides methods of treating or preventing a fire blight infection in a plant from the Rosaceae family comprising, expressing within the plant a recombinant peptide according to any of the foregoing aspects or embodiments.

In some embodiments, the plant from the Rosaceae family is an apple plant or a pear plant.

In some embodiments, expression of the recombinant peptide does not require alteration of the plant genome.

In another aspect, the present disclosure provides recombinant peptide according to any of the foregoing aspects or embodiments for use in treating or preventing a fire blight infection in a plant from the Rosaceae family. In some embodiments, the plant from the Rosaceae family is an apple plant or a pear plant.

In another aspect, the present disclosure provides uses of a recombinant peptide according to any of the foregoing aspects or embodiments for treating or preventing a fire blight infection in a plant from the Rosaceae family. In some embodiments, the plant from the Rosaceae family is an apple plant or a pear plant.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows the sequence of recombinant peptide 28P-2 and an alignment of the helical domains with a peptide from apple (Malus domestica).

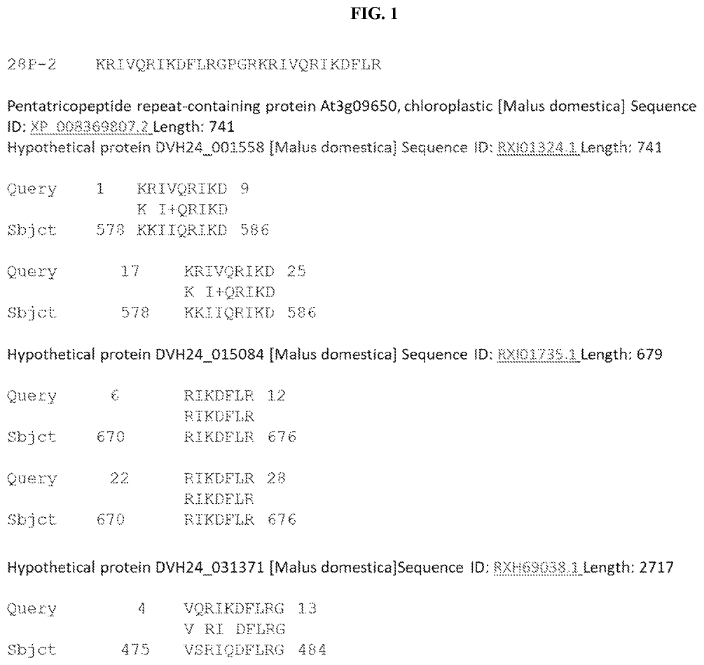

FIG. 2 shows the sequences of recombinant peptides 28P-4 and 28P-8, and an alignment of their respective helical domains with a peptide from apple (Malus domestica).

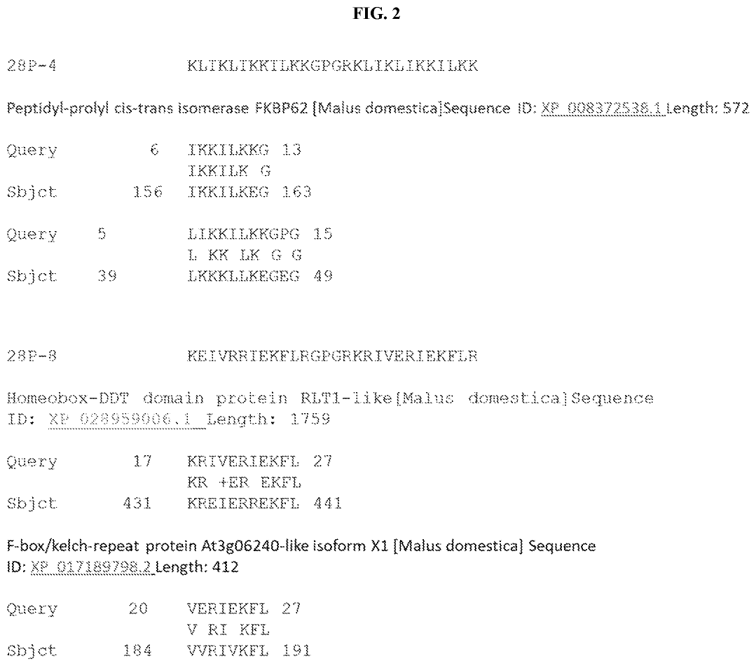

FIG. 3 shows recombinant peptides 28P-2, 28P-4, and 28P-2 and their respective minimum inhibitory concentrations (MIC). (A) shows an alignment of recombinant peptides 28P-2, 28P-4, and 28P-2. (B) shows the MIC of each of recombinant peptides 28P-2, 28P-4, and 28P-2 on Erwinia amylovora ATCC 49946.

FIG. 4 shows the sequence of recombinant peptide 36P and an alignment of the helical domains with a peptide from apple (Malus domestica).

FIG. 5 shows a comparison of the sequences and structures of 28P-2 and 36P and well as the MIC of both 28P-2 and 36P in killing several pathogenic microbes that affect tomato, apple, and pear plants. The MICs suggest that 36P is significantly more toxic to Erwinia amylovora than 28P-2.

FIG. 6 shows the bacterial primers used to detect Erwinia amylovora that were used to amplify the P29 gene.

FIG. 7 shows the Ct values of the P29 gene of Erwinia amylovora from leaf samples treated with water or 28P-2.

FIG. 8 shows the Ct values of Erwinia amylovora. The top panel shows Ct values that were measured for 100-10⁵ dilution of 10⁶ cfu/ml of the bacterium. The bottom panel shows a standard curve correlating cfu/ml and Ct values.

FIG. 9 shows the percent clearance of Erwinia amylovora by 28P-2 from individual tree samples utilized in the leaf dip assay.

FIG. 10 shows a side-by-side comparison of the Ct values for an apple reference gene, which remained unchanged after exposure to 28P-2.

### DETAILED DESCRIPTION

The present disclosure provides recombinant peptides comprising at least two helical peptide domains connected by a peptide linker. The present disclosure also provides methods of using the disclosed recombinant peptides to treat or prevent fire blight in apple and pear crops.

The disclosed recombinant peptides function by improving/supplementing the intrinsic innate immunity in plants, specifically apples and plants. The disclosed recombinant peptides can be used for treating or preventing various agricultural diseases, such as fire blight, which pose an imminent threat to the apple and pear industries around the world.

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a peptide” includes a single peptide as well as a plurality of peptides, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

As used herein, “effective amount” means the amount of a peptide that provides the specific pharmacological effect for which the compound (e.g., the disclosed chimeric peptides) is administered to a plant in need of such treatment or protection, i.e. to reduce, ameliorate, eliminate, clear, or prevent a fire blight infection or one or more signs or symptoms of the fire blight infection. It is emphasized that an effective amount of a compound will not always be effective in treating fire blight, even though such dosage is deemed to be an effective amount by those of skill in the art. The effective amount may vary based on the route of administration and formulation, the size of the plant, the bacteria being treated, and the severity of the infection, among other factors.

The terms “treatment” or “treating” as used herein with reference to plant or agricultural infections mean reducing, ameliorating, eliminating, or clearing a fire blight infection or one or more signs or symptoms of the fire blight infection.

The terms “prevent” or “protect” as used herein with reference to plant or agricultural infections mean blocking a fire blight infection from occurring in a plant or crop (e.g., apple or pear) that is at risk of infection or has been exposed to fire blight.

The term “fire blight” as used herein refers to an infection of a plant (preferably a plant of the Rosaceae family, such as apple or pear) by the bacterial pathogen Erwinia amylovora.

### II. Recombinant Peptides

Provided herein are novel recombinant peptides that possess antibacterial properties, among other beneficial activities. The disclosed peptides comprise at least two helical peptide domains connected by a peptide linker domain. The two helical peptide domains may be, for example, amphipathic helices. The disclosed peptide, which comprise a helix-turn-helix (HTH) structure, possess antimicrobial activity that results from the capacity to efficiently attach to, insert in, and rupture bacterial membranes.

a. Helical Peptide Domains

The at least two helical peptide domains may be isolated or derived from an endogenous plant protein or peptide. For example, many plants express amphipathic helical peptides that are capable of lysing viruses and bacteria as part of their innate immune system. Accordingly, the at least two helical peptide domains of the disclosed recombinant peptides may be isolated or derived from an apple plant, a pear plant, or any other crop or plant. In some embodiments, the at least two helical peptide domains are isolated or derived from the same type of plant that the peptide is intended to treat or protect (e.g., an antibacterial helical peptide from an apple or pear plant may be used to treat or prevent fire blight in apples or pears). In some embodiments, the at least two helical peptide domains are isolated or derived from a different type of plant than the peptide is intended to treat or protect (e.g., an antibacterial helical peptide from a plant other than an apple or pear plant may be used to treat or prevent fire blight in apples or pears).

The at least two helical peptide domains may each individually comprises 5-20, 8-18, 10-16, or 11-14 amino acids. For instance, each helical domain may independently comprise 5, 6, 7, 8, 9, 10, 11, 12, 13, 14, 15, 16, 17, 18, 19, or 20 amino acids. In some embodiments, the at least two helical peptide domains may each individually consists of 50, 45, 40, 37, 35, 34, 33, 32, 31, 30, 29, 28, 27, 26, 25, 20 or fewer amino acids. In some embodiments, each of the two helical peptide domains comprise the same number of amino acids. In some embodiments, each of the two helical peptide domains comprise a different number of amino acids. In some embodiments, each of the two helical peptide domains comprise the same amino acid sequence. In some embodiments, each of the two helical peptide domains comprise different amino acid sequences; for example, in some embodiments, one helical peptide sequence may be the mirror image or reverse of the other helical peptide sequence (e.g., KRIVQRIKDFLR (SEQ ID NO: 1) and RLFDKIRQVIRK (SEQ ID NO: 2)).

In some embodiments, the at least two helical peptide domains may comprises alternating nonpolar amino acid residues and positively charged amino acid residues. In some embodiments, the nonpolar residue is selected from the group consisting of glycine (G), alanine (A), valine (V), leucine (L), methionine (M), and isoleucine (I). In some embodiments, the nonpolar residue is selected from the group consisting of A, L, and I. In some embodiments, the nonpolar amino acid is selected from the group consisting of L and I. In some embodiments, the positively charged amino acid residue is selected from lysine (K), arginine (R), and histidine (H). In some embodiments, the positively charged amino acid residue is selected from K and R.

In some embodiments, any of the helical peptide domains may comprise an amino acid sequence consisting of 0-4 amino acid residues selected from the group consisting of polar uncharged residues, negatively charged residues, and nonpolar aromatic residues. In some embodiments, the helical peptide domains may each individually comprise 4, 3, 2, or 1 or fewer polar uncharged residues, negatively charged residues, and/or nonpolar aromatic residues. In some embodiments, the polar uncharged residues are selected from the group consisting of serine (S), threonine (T), cysteine (C), proline (P), asparagine (N), and glutamine (Q). In some embodiments, the negatively charged residues are selected from the group consisting of aspartate (D) and glutamate (E). In some embodiments, the nonpolar aromatic residues are selected from the group consisting of phenylalanine (F), tyrosine (Y), and tryptophan (W).

In some embodiments, a helical peptide domain may comprise a mixture of positively charged amino acid residues and nonpolar amino acid residues. In some embodiments, the ratio of positively charged amino acid residues to nonpolar amino acid residues is 0.7:1, 0.75:1, 0.8:1, 0.9:1, or 1:1. In some embodiments, the ratio of positively charged amino acid residues to nonpolar amino acid residues is 1.1:1, 1.2:1, 1.3:1, 1.4:1 and 15:1.

Helical peptide domains that are suitable for use as the disclosed recombinant peptides can be derived from agricultural crops including, but not limited to, apple plants and/or pear plants.

Table 1 provides exemplary helical peptide domains that have been derived from apple and pear plants or other agricultural crops (e.g., citrus or grape plants).

TABLE 1
Exemplary Helical Peptide Domains

| SEQ ID NO: | Sequence |
|---|---|
| 1 | KRIVQRIKDFLR |
| 2 | RLFDKIRQVIRK |
| 3 | KLIKLIKKILKK |
| 4 | KKLIKKILKILK |
| 5 | KEIVRRIEKFLR |
| 6 | RLFKEIRRVIEK |
| 7 | KRIVERIEKFLR |
| 8 | RLFKEIREVIRK |
| 9 | KRLVQRLKDFLR |
| 10 | RLFDKLRQVLRK |
| 11 | KRLIQRKRLIQR |
| 12 | RQILRKRQILRK |
| 13 | LIKLIKKILKK |
| 14 | KKLIKKILKIL |
| 15 | KKLAKEILKAL |
| 16 | LAKLIEKALKK |
| 17 | RRLIRRILRIL |
| 18 | LIRLIRRILRR |
| 19 | LIRLLRRILRR |
| 20 | RRLIRRLLRIL |
| 21 | LLIKLIKKILKK |
| 22 | KKLIKKILKILL |
| 23 | HPLIKLIKKILKK |
| 24 | KKLIKKILKILPH |
| 25 | GRLIKLIKKILKK |
| 26 | KKLIKKILKILRG |
| 27 | KLIRLIREILRR |
| 28 | RRLIERILRILK |
| 29 | KEIVRRIKEFLR |
| 30 | RLFEKIRRVIEK |
| 31 | LIKLCKKILKK |
| 32 | KKLIKKCLKIL |
| 33 | KVLSRVHAALKSIFDL |
| 34 | LDFISKLAAHVRSLVK |

The list of helical peptide domains provided in Table 1 is not intended to be limiting, and a skilled artisan would understand that other similar helical peptides can be derived from other plants and assessed for their ability to lyse bacterial membranes. Additionally, in some embodiments the disclosed recombinant peptides may comprise one or two helical peptide domains that are homologous to SEQ ID NOs: 1-34, but derived from other plants.

In some embodiments, a helical peptide domain of a disclosed recombinant peptide may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the helical peptides disclosed in Table 1.

In some embodiments, the helical domains of the disclosed recombinant peptides may not comprise the precise sequences of SEQ ID NOs: 1-34, but rather a consensus sequence that is homologous and maintains a helical structure. For example, in some embodiments, a helical domain may comprise at least 10, at least 11, at least 12, at least 13, at least 14, at least 15, at least 16, at least 17, or at least 18 amino acids and comprise a sequence of:
X¹X²X³X⁴X⁵X⁶X⁷X⁸X⁹X¹⁰X¹¹, wherein X¹, X², X⁴, X⁵, X⁸, and X⁹ are nonpolar residues, wherein X³, X⁶, X¹⁰, and X¹¹ are positively charged residues, and wherein X⁷ is a positively charged residue or negatively charged residue.
X¹X²X³X⁴X⁵X⁶X⁷X⁸X⁹X¹⁰X¹¹, wherein X², X⁵, X⁶, and X⁹ are positively charged residues, wherein X³, X⁴, X⁷, X⁸, X¹⁰ and X¹¹ are nonpolar residues, and wherein X¹ is a positively charged residue or negatively charged residue.
X¹X²X³X⁴X⁵X⁶X⁷X⁸X⁹X¹⁰X¹², wherein X¹, X², X⁶, X⁸, and X² are positively charged residues, wherein X³ and X⁴ are nonpolar residues, wherein X⁵ is a polar, uncharged residue, X⁷ is selected from a nonpolar residue and positively charged residue, X⁹ is a nonpolar residue or negatively charged residue, X¹⁰ is a nonpolar residue or nonpolar, aromatic residue, and X¹¹ is a nonpolar residue or a polar, noncharged residue.

In some embodiments, the nonpolar residue is selected from the group consisting of glycine (G), alanine (A), valine (V), leucine (L), methionine (M), and isoleucine (I). In some embodiments, the nonpolar residue is selected from the group consisting of A, L, and I. In some embodiments, the nonpolar amino acid is selected from the group consisting of L and I. In some embodiments, the positively charged amino acid residue is selected from lysine (K), arginine (R), and histidine (H). In some embodiments, the positively charged amino acid residue is selected from K and R.

In some embodiments, a helical domain may comprise at least 10, at least 11, at least 12, at least 13, at least 14, at least 15, at least 16, at least 17, or at least 18 amino acids and comprise a sequence of: (X¹ₙX²ₒ)ₚ, wherein X¹ is a nonpolar amino acid residue, X² is a positively charged amino acid residue, n is 1-3, o is 1-3, and p is 1-3; (X¹ₙX²ₒ)ₚ, or wherein X¹ is a positively charged amino acid residue, X² is a nonpolar amino acid residue, n is 1-3, o is 1-3, and p is 1-3. In some embodiments, at least one X¹ is selected from R and K. In some embodiments, at least one X² is selected from R and K. In some embodiments, at least one X² is selected from L and I.

b. Linker Domain

The flexible linker that connects the at least two helical domains is generally 2-50 amino acids in length and should allow the recombinant peptide, as a whole, to maintain a helix-turn-helix (HTH) structure. For instance, in some embodiments, the linker may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, the linker may be 3-15, 3-12, 3-5, 3-6, 4-10, 4-9, 4-8, 4-6, 4-5, 5-15, 5-10, 5-25, 10-20, 10-30, 10-40, or 15-25 amino acids in length. In some embodiments, the linker may be about 4 amino acids, at least 4 amino acids, or consist of 4 amino acids.

In some embodiments, the linker comprises 40-80% uncharged amino acid residues. In some embodiments, a helix domain disclosed herein comprises the linker comprises 35%, 40%, 45%, 50%, 55%, 60%, 65%, 70%, 75%, or 80% uncharged amino acid residues.

In some embodiments, the linker comprises 10-60% positively charged amino acid residues. In some embodiments, the linker comprises at least 5%, 6%, 7%, 8%, 9%, 10%, 11%, 12%, 13%, 14%, or 15% positively charged amino acid residues. In some embodiments, the linker comprises 60%, 55%, 50%, 45%, 40%, 35%, 30%, or fewer positively charged amino acid residues.

In some embodiments, the linker may comprise repeats of 1-3 or 2-3 amino acids such as, Glycine-Serine, Arginine-Tryptophan, Serine-Arginine-Aspartic Acid, etc. The linker may comprise a mixture of polar and nonpolar amino acids, for example, an alternating pattern of polar and nonpolar amino acids, and the ratio of polar to polar amino acids may be 1:1, 1:2, or 2:1.

Table 2 below provides exemplary linkers that are suitable for incorporation into the disclosed recombinant peptides.

TABLE 2
Exemplary Linkers

| SEQ ID NO: | Sequence |
|---|---|
| 35 | AAA |
| 36 | GGGSSGGGSG |
| 37 | GPGR |
| 38 | RDTPVVKS |
| 39 | AKDGIPAPTNYHKKHRAPVSCTGPAKM |
| 40 | GSTAPPA |
| 41 | RANATTLPKYYQNSRHPVSCTDPSK |
| 42 | RW |
| 43 | SRD |
| 44 | GSTAPPA |
| 45 | GS TAPP AGS TAPP A |
| 46 | QASHTCVCEFNCAPL |
| 47 | ARKKASIPNYYNSNLQPPVFCSDQSKM |
| 48 | YEQGAGRGSTAPPA |
| 49 | GSTA |
| 50 | GGGSGGGTDGR |

The list of linker peptides provided in Table 2 is not intended to be limiting, and a skilled artisan would understand that other similar peptides can be used to operably connect the at least two helical peptide domains.

In some embodiments, a linker peptide may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the linker peptides disclosed in Table 2.

c. Exemplary Recombinant Peptides

Recombinant peptides of the present disclosure may comprise any combination of two helical domains disclosed in Table 1 (or homologs thereof) connected by any of the linkers in Table 2 (or a homolog thereof), so long as the recombinant peptide can maintain a HTH structure.

In some embodiments, the two helical peptide domains are the same. In some embodiments, the helical peptide domains are different. For example, in some embodiments, the first helical peptide domain and the second helical peptide domain may be mirror images or the reverse of one another (e.g., KRIVQRIKDFLR (SEQ ID NO: 1) and RLFDKIRQVIRK (SEQ ID NO: 2)). In some embodiments, the two helical peptide domains may be isolated or derived from the same plant (e.g., apple or pear), while in some embodiments, the two helical peptide domains may be isolated or derived from different plants.

The disclosed recombinant peptides are about 20-50, about 20-45, about 20-40, about 20-35, about 20-30, about 20-25, about 25-50, about 25-45, about 25-40, about 25-35, about 25-30, about 30-50, about 30-45, about 30-40, or about 30-35 amino acids in length. For example, the recombinant peptide may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, the recombinant peptide is about 28 amino acids in length. In some embodiments, the recombinant peptide is about 36 amino acids in length. In some embodiments, each helical peptide domain may be about 10 to about 18 amino acids and the linker may be about 4 amino acids. In some embodiments, the linker is GPGR (SEQ ID NO: 37).

Table 3 below provides exemplary recombinant peptides that are suitable for treating fire blight in apple and pear plants.

TABLE 3
Exemplary Recombinant Peptides

| SEQ | |
|---|---|
| ID NO: | Sequence |
| 51 | KRIVQRIKDFLRGPGRKRIVQRIKDFLR [i.e., 28P-2] |
| 52 | KRIVQRIKDFLRGPGRRLFDKIRQVIRK |
| 53 | RLFDKIRQVIRKGPGRKRIVQRIKDFLR |
| 54 | RLFDKIRQVIRKGPGRRLFDKIRQVIRK |
| 55 | KLIKLIKKILKKGPGRKLIKLIKKILKK [i.e., 28P-4] |
| 56 | KLIKLIKKILKKGPGRKKLIKKILKILK |
| 57 | KKLIKKILKILKGPGRKLIKLIKKILKK |
| 58 | KKLIKKILKILKGPGRKKLIKKILKILK |
| 59 | KEIVRRIEKFLRGPGRKRIVERIEKFLR [i.e., 28P-8] |
| 60 | KEIVRRIEKFLRGPGRKEIVRRIEKFLR |
| 61 | KEIVRRIEKFLRGPGRRLFKEIRRVIEK |
| 62 | RLFKEIRRVIEKGPGRKRIVERIEKFLR |
| 63 | RLFKEIRRVIEKGPGRRLFKEIRRVIEK |
| 64 | KRIVERIEKFLRGPGRKEIVRRIEKFLR |
| 65 | KRIVERIEKFLRGPGRKRIVERIEKFLR |
| 66 | KRIVERIEKFLRGPGRRLFKEIREVIRK |
| 67 | RLFKEIREVIRKGPGRKEIVRRIEKFLR |
| 68 | RLFKEIREVIRKGPGRRLFKEIREVIRK |
| 69 | KRLVQRLKDFLRGPGRKRLVQRLKDFLR |
| 70 | KRLVQRLKDFLRGPGRRLFDKLRQVLRK |
| 71 | RLFDKLRQVLRKGPGRKRLVQRLKDFLR |
| 72 | RLFDKLRQVLRKGPGRRLFDKLRQVLRK |
| 73 | KRLIQRKRLIQRGPGRKRLIQRKRLIQR |
| 74 | KRLIQRKRLIQRGPGRRQILRKRQILRK |
| 75 | RQILRKRQILRKGPGRKRLIQRKRLIQR |
| 76 | RQILRKRQILRKGPGRRQILRKRQILRK |
| 77 | LIKLIKKILKKGPGRLIKLIKKILKK |
| 78 | LIKLIKKILKKGPGRKKLIKKILKIL |
| 79 | KKLIKKILKILGPGRLIKLIKKILKK |
| 80 | KKLIKKILKILGPGRKKLIKKILKIL |
| 81 | KKLAKEILKALGPGRKKLAKEILKAL |
| 82 | KKLAKEILKALGPGRLAKLIEKALKK |
| 83 | LAKLIEKALKKGPGRKKLAKEILKAL |
| 84 | LAKLIEKALKKGPGRLAKLIEKALKK |
| 85 | RRLIRRILRILGPGRRRLIRRILRIL |
| 86 | RRLIRRILRILGPGRLIRLIRRILRR |
| 87 | LIRLIRRILRRGPGRRRLIRRILRIL |
| 88 | LIRLIRRILRRGPGRLIRLIRRILRR |
| 89 | LIRLLRRILRRGPGRLIRLLRRILRR |
| 90 | LIRLLRRILRRGPGRRRLIRRLLRIL |
| 91 | RRLIRRLLRILGPGRLIRLLRRILRR |
| 92 | RRLIRRLLRILGPGRRRLIRRLLRIL |
| 93 | LLIKLIKKILKKGPGRLLIKLIKKILKK |
| 94 | LLIKLIKKILKKGPGRKKLIKKILKILL |
| 95 | KKLIKKILKILLGPGRLLIKLIKKILKK |
| 96 | KKLIKKILKILLGPGRKKLIKKILKILL |
| 97 | HPLIKLIKKILKKGPGRHPLIKLIKKILKK |
| 98 | HPLIKLIKKILKKGPGRKKLIKKILKILPH |
| 99 | KKLIKKILKILPHGPGRHPLIKLIKKILKK |
| 100 | KKLIKKILKILPHGPGRKKLIKKILKILPH |
| 101 | GRLIKLIKKILKKGPGRGRLIKLIKKILKK |
| 102 | GRLIKLIKKILKKGPGRKKLIKKILKILRG |
| 103 | KKLIKKILKILRGGPGRGRLIKLIKKILKK |
| 104 | KKLIKKILKILRGGPGRKKLIKKILKILRG |
| 105 | KLIRLIREILRRGPGRKLIRLIREILRR |
| 106 | KLIRLIREILRRGPGRRRLIERILRILK |
| 107 | RRLIERILRILKGPGRKLIRLIREILRR |
| 108 | RRLIERILRILKGPGRRRLIERILRILK |
| 109 | KEIVRRIKEFLRGPGRKEIVRRIKEFLR |
| 110 | KEIVRRIKEFLRGPGRRLFEKIRRVIEK |
| 111 | RLFEKIRRVIEKGPGRKEIVRRIKEFLR |
| 112 | RLFEKIRRVIEKGPGRRLFEKIRRVIEK |
| 113 | LIKLCKKILKKGPGRLIKLCKKILKK |
| 114 | LIKLCKKILKKGPGRKKLIKKCLKIL |
| 115 | KKLIKKCLKILGPGRLIKLCKKILKK |
| 116 | KKLIKKCLKILGPGRKKLIKKCLKIL |
| 117 | KVLSRVHAALKSIFDLGPGRKVLSRVHAALKSIFDL [36P] |
| 118 | KVLSRVHAALKSIFDLGPGRLDFISKLAAHVRSLVK |
| 119 | LDFISKLAAHVRSLVKGPGRKVLSRVHAALKSIFDL |
| 120 | LDFISKLAAHVRSLVKGPGRLDFISKLAAHVRSLVK |

The list of recombinant peptides provided in Table 3 is not intended to be limiting, and a skilled artisan would understand that other similar peptides can be used to treat fire blight in apple and pear plants.

In some embodiments, a recombinant peptide may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the recombinant peptides disclosed in Table 3.

The disclosed recombinant peptides may be produced by known means in the art such as, for example, recombinant expression in a suitable host cell. In some embodiments, the disclosed peptides may be expressed by human embryonic kidney (HEK) cells or Chinese hamster ovary (CHO) cells or any other available cell expression system. Alternatively, the peptides may be expressed recombinantly by or in the plant for which protection or treatment is desired.

### III. Formulations of the Disclosed Recombinant Peptides

Formulations suitable for use in the methods described herein can be formulated with one or more of the disclosed recombinant peptides and an acceptable carrier or diluent. The content of the recombinant peptides within a formulated composition may be from about 0.01 to about 95%. The recombinant peptides may be formulated into various types of compositions, including but not limited to an oil solution, emulsifiable concentrate, wettable powder, flowable (aqueous suspension or aqueous emulsion), granule, dust and so on, by mixing with solid carrier, liquid carrier, or gaseous carrier and optionally surfactant, the other formulation additive.

Non-limiting examples of solid carriers that can be used in a formulation comprising the disclosed recombinant peptides include inorganic carriers such as clays (e.g., kaolin clay, diatomaceous earth, synthetic hydrated silicon oxide, bentonite, Fubasami clay, acid clay), talc, ceramics, sericite, quartz and calcium carbonate. Examples of the liquid carrier include water, alcohols (e.g., methanol, ethanol, higher alcohols), ketones (e.g., acetone, methyl ethyl ketone), aromatic hydrocarbons (e.g., benzene, toluene, xylene, ethylbenzene, methylnaphthalene), aliphatic hydrocarbons (e.g., hexane, cyclohexane, kerosene, gas oil), esters (ethyl acetate, butyl acetate), nitrites (e.g., acetonitrile, isobutyronitrile), ethers (e.g. diisopropyl ether, dioxane), acid amides (e.g., N,N-dimethylformamide, N,N-dimethylacetamide), halogenated hydrocarbons (e.g., dichloromethane, trichloroethane, carbon tetrachloride), dimethyl sulfoxide and vegetable oils (e.g., soybean oil, cottonseed oil). Examples of the liquefied gaseous carrier include fluorocarbon, fluorohydrocarbon, LPG (liquefied petroleum gas), dimethyl ether and carbon dioxide.

Non-limiting examples of the surfactant optionally used in the disclosed formulations can include alkyl sulfate salts, alkylsulfonate salts, alkylarylsulfonate salts, alkyl aryl ethers, polyoxyethylenealkyl aryl ethers, polyethylene glycol ethers, polyhydric alcohol esters and sugar alcohol derivatives.

The other formulation auxiliaries are exemplified by sticking agents, dispersants, and stabilizers. Non-limiting examples of sticking agents and dispersants include casein, gelatin, polysaccharides (e.g., starch powder, gum arabic, cellulose derivatives, alginic acid), lignin derivatives, bentonite, sugars and synthetic water-soluble polymers (e.g., polyvinyl alcohol, polyvinylpyrrolidone, polyacrylic acids). Non-limiting examples of stabilizer include phenol type antioxidants such as BHT (2,6-di-tert-butyl-4-methyphenol) and BHA (mixture of 2-tert-butyl-4-methoxyphenol and 3-tert-butyl-4-methoxyphenol), amine type antioxidants such as diphenylamine, organic sulfur type antioxidants such as 2-mercaptobenzimidazole, PAP (acid isopropyl phosphate), vegetable oils, mineral oils, surfactants, fatty acids and esters of fatty acid.

Flowable formulations (aqueous suspension or aqueous emulsion) may comprise one or more of the disclosed recombinant peptides, a dispersant, a suspension assistant (for example, protective colloid or a compound giving thixotropy), suitable auxiliaries (for example, antifoamer, rust preventive agent, stabilizer, developing agent, penetrating assistant, antifreezing agent, bactericide, fungicide, etc.) and water. Non-limiting examples of a protective colloid include gelatin, casein, gums, cellulose ethers and polyvinyl alcohol, and examples of the compound giving thixotropy include bentonite, aluminum magnesium silicate, xanthan gum and polyacrylic acids. Use of an oil, which can, in some instance, solubilize a disclosed recombinant peptide, in place of water can provide a suspension-in-oil formulation.

The formulations of emulsifiable concentrate, wettable powder, flowable and so on obtained above may be diluted with water or another suitable vehicle, and applied at 0.1 to 10000 ppm of the concentration of the recombinant peptides. The formulations of oil solution, granule, dust and so on are may be applied to an intended plant, seed, trunk, or leaf directly as they are.

In some embodiments, a mixture of one or more of the disclosed recombinant peptides or a liquid formulation thereof and a propellant can be charged into a pressure container with a spray nozzle to afford an aerosol of the disclosed recombinant peptides. Non-limiting examples of the propellant for aerosols include propane, butane, isobutane, dimethyl ether, methyl ethyl ether and methylal.

In some embodiments, rather than applying one or more of the disclosed recombinant peptides to a specific plant, the recombinant peptide(s) may be applied (e.g., sprayed or otherwise dispersed) in a general target area where it is desirable to prevent the spread of a particular disease (e.g., fire blight) or treat a diseased population of plants. The target area may be, for example, a site of a known infection or a field where crops are being grown.

The application amount and concentration of the disclosed recombinant peptides that should be applied to a given plant or target area can be suitably designed according to the type of the formulations, time, place, and method of application, kind of target plant, and the type of use desired (e.g., treatment or prevention).

### IV. Methods of Using the Disclosed Recombinant Peptides to Treat and Prevent Fire Blight

The disclosed recombinant peptides may be useful for a variety of agricultural applications, but as disclosed herein, are particularly useful in treating or preventing fire blight in plants of the Rosaceae family, and in particular apple and pear crops. Fire blight affects plants across the Rosaceae family, which includes trees and shrubs in orchards, nurseries and landscape plantings. The plants affected include Amelanchier (serviceberry), Chaenomeles (flowering quince), Cotoneaster (cotoneaster), Crataegus (hawthorn), Eriobotrya (loquat), Malus (apple and crabapple), Photinia (photinia), Prunus (flowering almond, plum and cherry), Pyracantha (pyracantha), Pyrus (pear), Rosa (rose), and Spirea (spirea). Any of these host plants may be treated according to the following methods utilizing the disclosed recombinant peptides.

In one aspect, the present disclosure provides methods of using one or more of the disclosed recombinant peptides to treat or prevent a fire blight infection in a plant (e.g., an apple or pear plant). The one or more recombinant peptides may be the same (i.e., comprise the same helical domains and linker domains or different (i.e., comprise one or more distinct helical or linker domains). The one or more recombinant peptides may be administered to a plant, a target site on a plant, or a target site near a plant (e.g., the dirt around the roots or a fence, arbor, or other structure on which a vine is growing or intended to grow) or where a plant is intended to be cultivated (e.g., a field prior to planting a crop). Additionally or alternatively, the one or more recombinant peptides may be expressed by the target plant that is intended for treatment or protection from fire blight by introducing a gene or expression vector encoding the one or more peptides into the target plant.

The disclosed recombinant peptides improve on the innate antimicrobial defense of a target plant by utilizing a plant-derived amphipathic helical lytic peptides to penetrate the bacterial membrane and destroy the bacteria via lysis. Many bacteria possess membranes that may be lysed by a recombinant peptide as disclosed herein, and given the non-specific nature of this mechanism of action, the disclosed recombinant peptides are believed to possess broad spectrum antibacterial activity, but are particularly effective against fire blight.

Accordingly, the disclosed methods of treating or preventing fire blight infection in a target plant can be applied to treating or preventing an infection in a plant from the Rosaceae family. In some embodiments, the plant from the Rosaceae family is an apple plant or a pear plant. The disclosed methods of treatment and prevention are applicable to all cultivars of appeals and all cultivars of pears, as these respective cultivars share significant genetic homology. For examples, the disclosed methods are useful in treating apple cultivars including, but not limited to, Red Delicious, Golden Delicious, Johnathan, Jonagold, Gala, Honeycrisp, Braeburn, cripps, Rome, Cox's Orange Pippin, Idared, Mutsu, Elstar, Cosmic Crisp, Ginger Gold, Cortland, Paula Red, Zestar, Winesap, Northern Spy, Pink Lady, Pacific Rose, Fuji, Tentation (Delblush), Ambrosia, Piñata, Jazz (SciFresh), Sundowner, Envy, SweeTango, and Aurora. The disclosed methods are useful in treating apple cultivars including, but not limited to, Williams, Bosc, D'Anjou, Comice, Abate Fetel, Conference, Winter Nelis, Red Anjou, Green Anjou, Bartlett, Red Bartlett, Concorde, Forelle, Seckel, and Starkrimson.

For the purposes of the disclosed methods, a recombinant peptide may be applied to a plant (e.g., apple or pear plant), seed, or portion of a plant three or more times a day, twice a day, or once a day. In some embodiments, the recombinant peptide may be applied once a day, once every other day, three times a week, twice a week, once a week, once every other week, once every three weeks, once a month, once every other month, once every three months, once every four months, once every five months, once every six months, or less frequently. In such embodiments, the recombinant peptide may be applied to a plant, either sequentially or concurrently, with one or more additional insecticides, larvicides, fungicides, antibiotics, anti-microbials, herbicides, or arthropod repellents.

For the purposes of the disclosed methods, the recombinant peptides be applied to the intended plant (e.g., apple or pear plant), seed, or portion of a plant in any appropriate form, such as in a spray, aerosol, liquid, gel, powder, or solid form. The recombinant peptides may be formulated and applied to a plant, seed, or portion of a plant as solids, liquids, or gases (e.g., using a vapor delivery system). Alternatively or additionally, one or more expression vectors encoding the disclosed recombinant peptides may be introduced into the plant (e.g., apple or pear plant) via transgenic or non-transgenic techniques such that the desired recombinant peptides are expressed by the target plant to prevent or treat fire blight.

For example, the recombinant peptides described herein can be applied via a number of formulation types, including isolated recombinant peptides, which may further be coupled with dustable powders (DP), soluble powders (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed/plant treatment formulations.

In some embodiments, delivery of the recombinant peptides to plants (e.g., apple or pear plants) can be via different routes. The recombinant peptides can be suitably administered as an aerosol, for example by spraying onto leaves or other plant material. The recombinant peptides can also be administered by injection, for example directly into a plant, such as into the stem. In certain embodiments the recombinant peptides are administered to the roots. This can be achieved by spraying or watering plant roots with compositions. In some embodiments, the recombinant peptides are introduced into the xylem or phloem, for example by injection or being included in a water supply feeding the xylem or phloem. Application to the stems or leaves of the plant can be performed by spraying or other direct application to the desired area of the plant; however, any method known in the art can be used. A solution or vehicle containing the recombinant peptides at a dosage of active ingredient can be applied with a sprayer to the stems or leaves until runoff to ensure complete coverage, and repeat three or four times in a growing season. The concentrations, volumes and repeat treatments may change depending on the plant, route of administration, and the fire blight being treated or prevented.

Additional embodiments of the invention include a polynucleotide comprising a nucleic acid sequence that may encode one or more of the recombinant peptides described herein. For example, some embodiments may include a polynucleotide comprising a nucleic acid sequence that encodes one of more of SEQ ID NOs: 51-120 or a homolog that possesses about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any one of SEQ ID NOs: 51-120. Such sequences may further be operably linked to a promotor to generate an expression vectors and further introduced to a plant, for example, an apple or pear plant. In this embodiment, such transformed plant or plant cell may produce the recombinant peptide(s) in vivo. Such a transformed plant may exhibit enhanced resistance to any number of bacterial or viruses. In some embodiments, a transformed plant may exhibit decreased bacterial loads and/or decreased symptoms or progression of fire blight or other bacterial or viral diseases. Methods, systems and techniques of stable and transient plant transformation, such as Agrobacterium tumefaciens-mediated transformation, carbon nanotube (CNT)-based transformation, and the like are known in the art and included within the scope of the present disclosure.

Briefly, transformation methods for expressing the recombinant peptides in vivo within with plants (e.g., apples or pear plants) targeted for treatment or protection may include, but are not limited to, in planta methods the delivery of the recombinant peptide genes that do not involve alteration of the plant (e.g., apple or pear) genome (i.e., non-GMO routes), such as carbon nanotube (CNT) and non-infectious GLRaV-7 based delivery systems.

For the purposes of a CNT-based approach, single wall COOH-CNT can be linked to positively charged poly-ethylen-imine (PEI), on which high copy pUC plasmid DNA encoding one or more of the recombinant peptides can be bound by electrostatic interactions. The gene encoding the recombinant peptide contain an N-terminal secretion signal and upstream 2X³⁵S CaMV and downstream NOS terminator. CNT-PEI-DNA may be about 10 to about 20 nm in length to allow the nanoparticle to penetrate the extracellular matrix and enter the plant cell. CNT-PEI-DNA particles can be introduced into a target plant by various routes, such as injection into the leaves by needleless syringes. Following expression of the transformed recombinant peptide(s) within the target plant, the protein will exert antibacterial activity.

For the purposes of a non-infectious GLRaV-7 route, agrobacterium containing a binary vector harboring GLRaV-7 with one or more genes encoding one or more of the disclosed recombinant peptides can be infiltrated in the leaves. This allows a sustained in planta delivery and transport of the recombinant peptide throughout the plant or in a targeted tissue of the plant. The viral gene cassette and the recombinant gene can be driven by, for example, a 2X³⁵S CaMV promoter and NOS terminator.

The following examples are given to illustrate the present invention. It should be understood, however, that the invention is not to be limited to the specific conditions or details described in these examples.

### EXAMPLES

### Example 1—Minimum Inhibitory Concentrations of Exemplary Recombinant Peptides

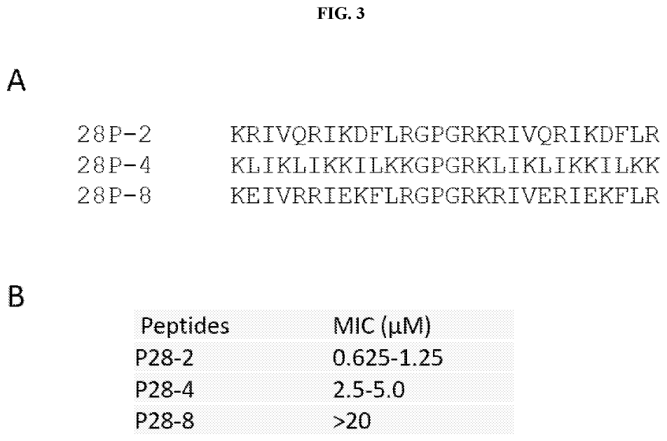

Minimum inhibitory concentrations were calculated for recombinant peptides 28P-2, 28P-4, and 28P-8 (FIG. 3A) by exposing Erwinia amylovora (ATCC 49946) to serial dilutions of these exemplary recombinant peptides. These assays showed that all three peptides were active against the causative pathogen of fire blight with 28P-2 being the most active 28-amino acid-long recombinant peptide that was tested (FIG. 3B).

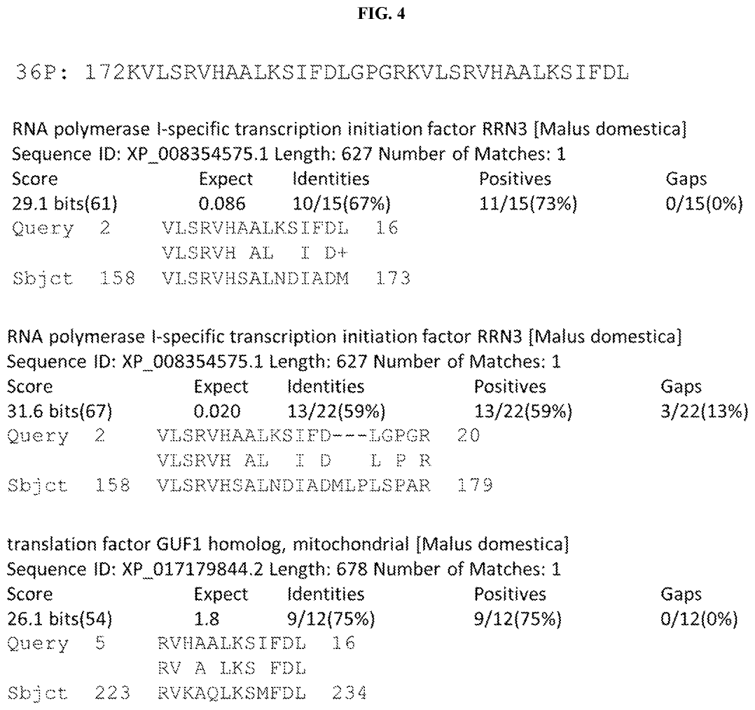

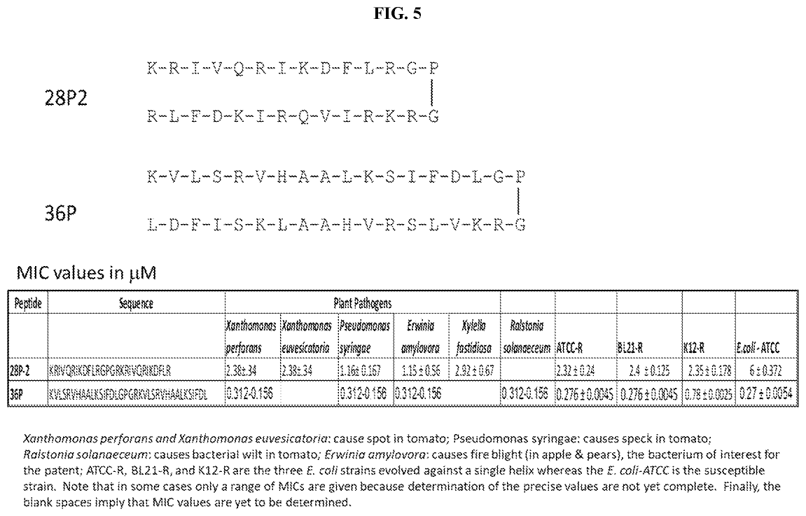

Beyond the 28-amino acid-long recombinant peptides 28P-2, 28P-4, and 28P-8, a 36-amino acid-long recombinant peptide was also prepared and tested. This peptide—referred to as 36P (see FIG. 4)—shows significant activity against Xanthomonas perforans and Xanthomonas euvesicatoria, which cause spot in tomato, Pseudomonas syringae, which causes speck in tomato, and Ralstonia solanaeceum, which causes bacterial wilt in tomato, in addition to Erwinia amylovora. Additionally, ATCC-R, BL21-R, and K12-R are the three E. coli strains that have evolved resistance against naturally occurring single helix, but these were susceptible to the disclosed HTH recombinant peptides. FIG. 5 shows the results of the assays used to determine the MICs of 28P-2 and 36P for these microbes. Note that in some cases only a range of MICs are given because determination of the precise values are not yet complete. The blank spaces imply that MIC values are yet to be determined.

### Example 2—Detached Leaf Assays

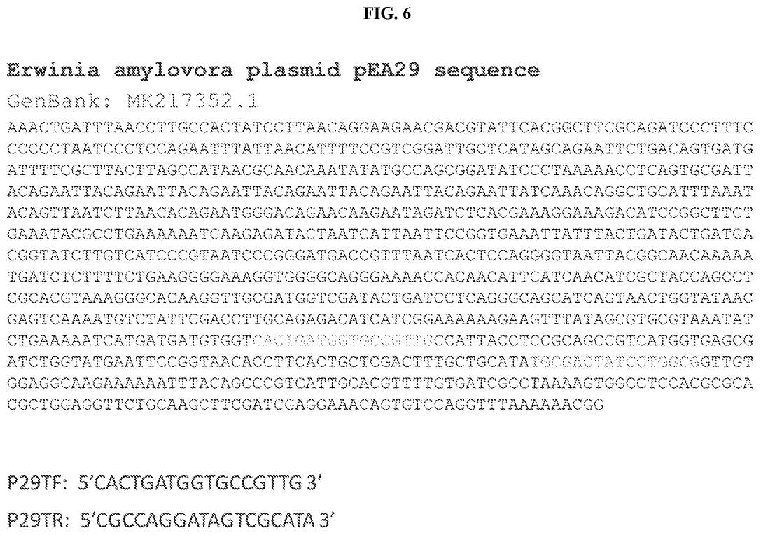

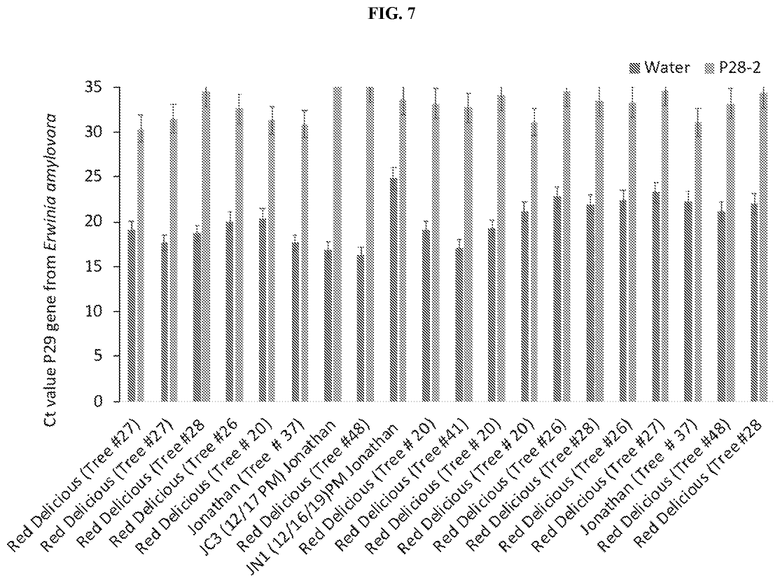

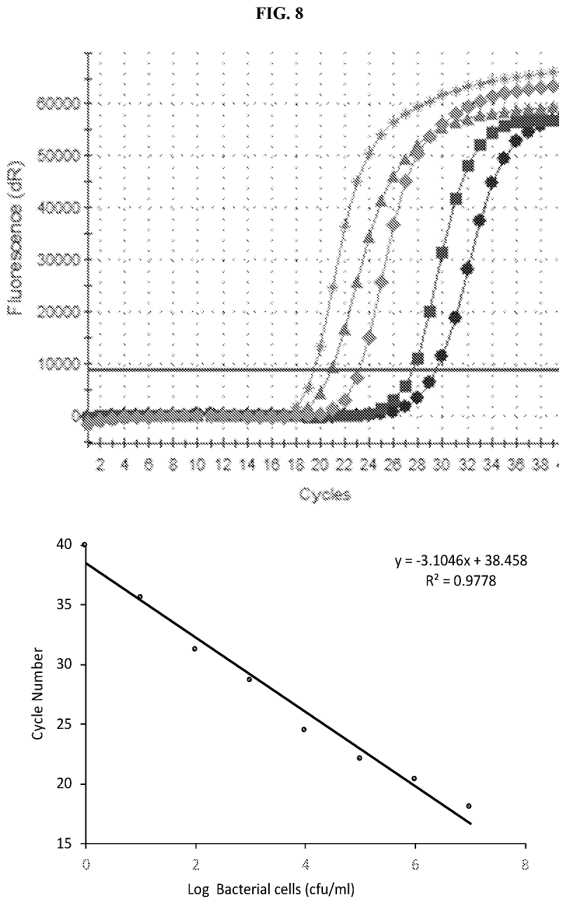

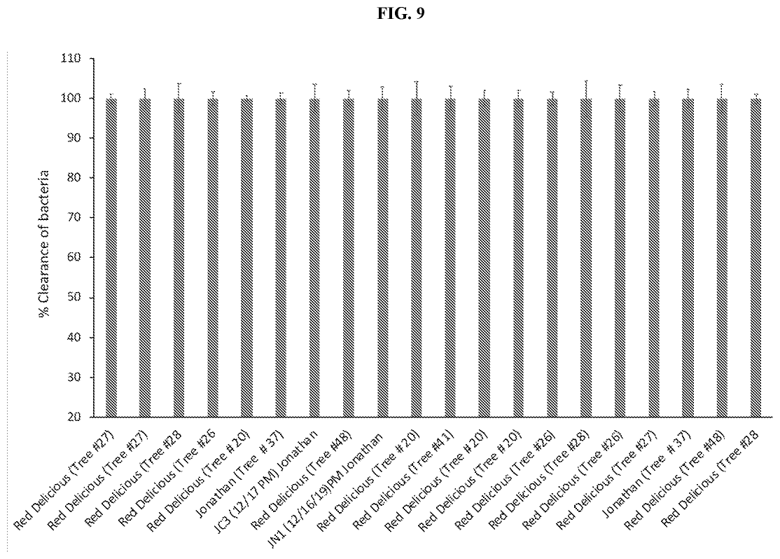

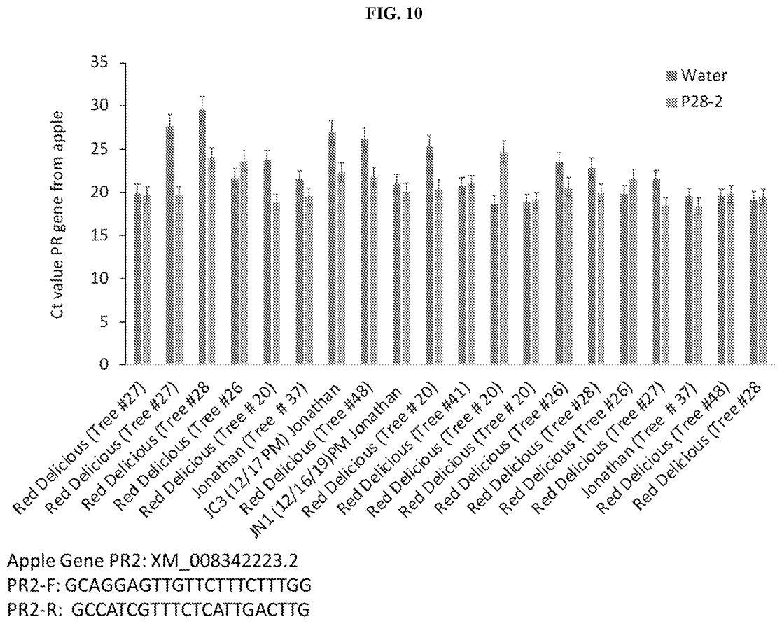

Detached leaf assays on infected apple varieties Jonathan and Red Delicious showed 100% clearance of Erwinia amylovora by 20 mM 28P-2. Water was used as a control. The infected leaves were dipped in 1 ml of the peptide solution for 4 hours to allow the solution to be absorbed. The genomic DNA was extracted and analyzed by qPCR. Bacteria specific primers were used to measure the Ct values (FIG. 6), the higher the Ct value the lower is the bacterial. Upon treatment, the Ct values are increased, and thus the treatment with 28P-2 was shown to be effective at killing Erwinia amylovora. See FIG. 7. The Ct values were also measured 100-10⁵ dilution of 10⁶ cfu/ml of the bacterium (FIG. 8, top). This gives a standard curve correlating cfu/ml and Ct values (FIG. 8, bottom). FIG. 9 shows the 100% clearance of Erwinia amylovora by 28P-2. The Ct values were also measured for an apple reference gene, the level of which remained unchanged upon treatment (FIG. 10) indicating bacterial clearance and not a global reduction in nucleic acids. Thus, the results of the leaf dip assay show that 28P-2 is effective in treating Erwinia amylovora without harming or altering the expression of genes in the treated apple plant.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

1. A recombinant peptide comprising at least two helical peptide domains connected by a linker domain, wherein the at least two helical domains were isolated or derived from an apple or pear plant.

2. The recombinant peptide of claim 1, wherein the at least two helical domains are about 10 to about 20 amino acids in length.

3. The recombinant peptide of claim 1, wherein the linker domain is about 4 amino acids.

4. The recombinant peptide of claim 1, wherein the recombinant peptide comprises 28 amino acids or 36 amino acids.

5. The recombinant peptide of claim 1, wherein each helical peptide domain is independently selected from any one of SEQ ID NOs: 1-34.

6. The recombinant peptide of claim 1, wherein the at least two helical peptides comprise the same amino acid sequence.

7. The recombinant peptide of claim 1, wherein the at least two helical peptides comprise different amino acid sequences.

8. The recombinant peptide of claim 1, wherein the linker domain comprises SEQ ID NO: 37.

9. The recombinant peptide of claim 1, wherein the recombinant peptide comprises any one of SEQ ID NOs: 51-120.

10. The recombinant peptide of claim 1, wherein the recombinant peptide comprises an amino acid sequence that comprises at least about 80% homology to any one of SEQ ID NOs: 51-120.

11. The recombinant peptide comprising an amino acid sequence selected from SEQ ID NO: 51, SEQ ID NO: 55, SEQ ID NO: 59, and SEQ ID NO: 117.

12. A formulation comprising a recombinant peptide according to claim 1 and an acceptable carrier or diluent, wherein the carrier is a solid, a liquid, a spray, or an aerosol.

13-15. (canceled)

16. A method of treating or preventing a fire blight infection in a plant from the Rosaceae family comprising, applying to a target area on or adjacent to a plant from the Rosaceae family an effective amount of a recombinant peptide comprising two helical peptide domains connected by a linker domain, wherein the two helical domains are about 10 to about 20 amino acids in length and the linker domain is about 4 amino acids.

17. The method of claim 16, wherein the target area comprises a plant, the seed of a plant, or a portion of the plant.

18. The method of claim 16, wherein the plant from the Rosaceae family is an apple plant or a pear plant.

19. The method of claim 16, wherein the target area is the soil in which a plant from the Rosaceae family is growing, a field that will be planted, or a structure on which a plant is growing.

20. The method of claim 16, wherein applying comprises spraying the target with the recombinant peptide.

21. A method of treating or preventing a fire blight infection in a plant from the Rosaceae family comprising, expressing within the plant a recombinant peptide comprising two helical peptide domains connected by a linker domain, wherein the two helical domains are about 10 to about 20 amino acids in length and the linker domain is about 4 amino acids.

22. The method of claim 21, wherein the plant from the Rosaceae family is an apple plant or a pear plant.

23. The method of claim 21, wherein expression of the recombinant peptide does not require alteration of the plant genome.

24-27. (canceled)
